(19) United States
(12) Patent Application Publication
(10) Pub. No.: US 2009/0040461 A1
(43) Pub. Date: Feb. 12, 2009

(54) LOW VISION AID DEVICE

(75) Inventors: Uzi Efron, Herzliya (IL); Yizhak David, Holon (IL); Boris Apter, Ashdod (IL); Nonel Thirer, Hod Hasharon (IL); Itzhak Baal-Zedaka, Rosh Ha'Ayin (IL)

Correspondence Address: OBLON, SPIVAK, MCCLELLAND MAIER & NEUSTADT, P.C., 1940 DUKE STREET, ALEXANDRIA, VA, 22314, US

(73) Assignees: BEN GURION UNIV. OF THE NEGEV RESEA. & DEV. AUTH., BEER SHEVA (IL); A.Y.Y.T.-TECH. APPLICATIONS & DATA UPDATE LTD., HOLON (IL)

(21) Appl. No.: 11/910,490

(22) PCT Filed: Apr. 2, 2006
(86) PCT No.: PCT/IL2006/000420

§ 371 Date: May 13, 2008

(30) Foreign Application Priority Data
Apr. 3, 2005 (IL) 167821

(51) Int. Cl. A61B 3/14

(52) U.S. Cl. 351/210

(57) ABSTRACT

There is provided a low-vision aid device, including a scene-display imager producing a signal pattern composed of an array of pixels, a near IR illuminator for illuminating the retina of the eye with radiation for eye tracking, to be reflected from the retina of the eye, an eye-retina tracking imager, and an image transceiver device capable of providing both functions of eye imaging as well as image display by selectively rotating the polarization of individual pixels of the array of pixels of the signal pattern, to allow the transference of selected portions of the signal pattern to reach the retina of the eye.

### FIELD OF THE INVENTION

The present invention relates to a low-vision aid device and more particularly to an image transceiver device (ITD), for retinal re-mapping for visual enhancement.

### BACKGROUND OF THE INVENTION

Between 2 to 4% of the total population and up to 60% of our aging population suffer from vision loss not readily correctable with ordinary optical or medical intervention. People with low vision typically have significantly reduced visual acuity and a significant loss of contrast sensitivity, often in combination with visual field loss. These impairments cause a number of disabilities including difficulty with reading, writing, recognizing faces, orientation and mobility, and other activities of daily life. Thus, the partial or complete loss of vision acuity in the central (fovial) region of the retina caused by aged-related macular degeneration (AMD) will deprive people of their ability to read, discriminate objects or recognize faces. The loss of peripheral vision caused for example by retinitis pigmentosa (RP) results in the loss of the peripheral field, which is critically needed for orientation and mobility. Other local vision losses will result in the appearance of visual “holes” or scotomas, where persons lose partial areas of their visual field of view.

While there exist a variety of low vision aid instruments, they are almost all limited to static optical correction, e.g., mini-telescope mounted on eye-glasses, whereas the preferred solution calls for an adaptive correction including pre-processing and conditioning of the input imagery. There also exists no practical solution for those low vision persons who experience mobility problems.

Recently there have been several attempts to provide some measure of basic Image conditioning in mobile, head-mounted devices, however these devices fall quite short of the required solution in that they fail to provide:
a) the necessary image re-mapping and image processing;
b) See-through capability; and
c) Compact size head-mounted gear.

It should be emphasized that the issue of the appearance or cosmetics of the aid is of a paramount importance. That is why the large, bulky head mounted (HM) goggle, while providing better functionally, is usually unacceptable.

It is obvious from the above discussion that the solution sought must be an integrated one, comprised of an efficient compact HM optics, combined with adaptive electro-optic devices, capable of executing the required image pre-processing and retinal-re-direction functions in a compact, eye-glasses-size goggle.

The present invention is therefore centered around the use of a novel image transceiver device (ITD) chip allowing both functions of imaging and display to be implemented in a single chip which, when mounted on a properly-designed HM gear allows the necessary imaging and display functions to be implemented in a compact, eyeglasses-size, low-vision (LV) goggle.

### DISCLOSURE OF THE INVENTION

A broad object of the invention is to provide a device using an imager part of the ITD device to acquire the required field-of view (FOV) and then use the display part to direct the processed imagery to the healthy, undamaged part of the retina of an eye.

It is a further object of the present invention to provide a device for retinal re-mapping and image processing and enhancement for displaying the processed image to the healthy part of the retina of the eye.

In accordance with the present invention there is therefore provided a low-vision aid device, comprising a scene-display imager producing a signal pattern composed of an array of pixels; means for illuminating the retina of said eye with near IR (NIR) radiation for eye tracking, to be reflected from the retina of the eye; an eye-retina tracking imager, and an image transceiver device (ITD) having means capable of providing both functions of eye imaging as well as image display by selectively rotating the polarization of individual pixels of said array of pixels of the signal pattern, to allow the transference of selected portions of said signal pattern to reach the retina of the eye.

Thus, the present invention provides an ITD which combines the display function with either one of the imaging functions or, with both of them simultaneously, to form a three-function device, thereby significantly reducing the Low-Vision goggle size of a user.

### BRIEF DESCRIPTION OF THE DRAWINGS

The invention will now be described in connection with certain preferred embodiments with reference to the following illustrative figures, so that it may be more fully understood.

With specific reference now to the figures in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic optical illustration of a low vision-aid device, according to the present invention;

FIG. 2 is a cross-sectional view of a preferred embodiment of an imager/display ITD according to the present invention, and

FIGS. 3 and 4 are further embodiments of a low vision aid device according to the present invention.

### DETAILED DESCRIPTION OF THE PREFERRED EMBODIMENTS

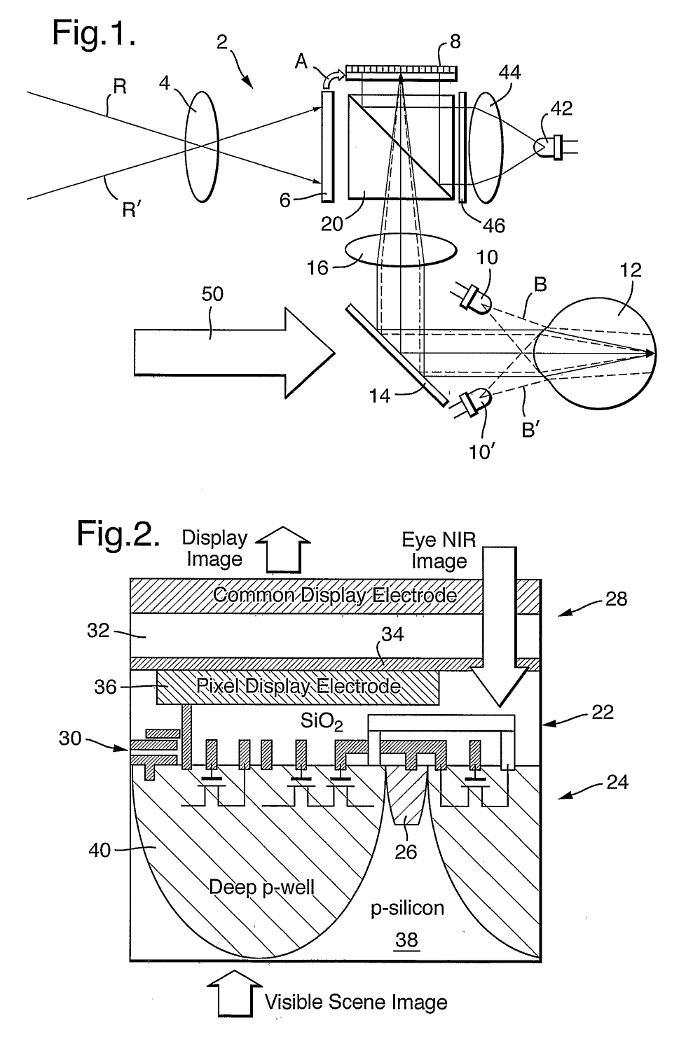

Referring to FIG. 1, there is illustrated an embodiment of a low-vision aid device 2 according to the present invention. Starting with the two crossed rays R, R′, which indicate the observed scenery, after passing a scene-imager optics 4, is imaged by a scene imager 6, which can be constituted by a simple CMOS imager. Following appropriate image processing of the scene information, the processed image is transferred, as indicated by the arrow A, to the display part of the eye-tracker(imager)/display (image transceiver device) ITD 8 for display. The NIR light emitting diodes (NIR-LEDs) 10, 10′, located adjacent to the viewer's eye 12, radiate R-polarized NIR beams B, B′ into the observer's eye 12. The R-polarized NIR beams are reflected from the retina, carrying the retinal image down the optical path, as shown by the broken lines. The image beam is then reflected by the non-polarizing beam splitter (NBS) 14, passes through a display eyepiece/eye tracker optics 16 to a polarizing beam splitter (PBS) 20, e.g., a cube, which transmits R-polarization, but reflects S-polarization into the imager part of the ITD 8.

For better understanding, reference is now made to FIG. 2 illustrating a preferred embodiment of an imager/display ITD 8 in the form of a chip 22. The chip (single-pixel structure shown), consists of an imaging part 24 with a photodiode 26) and the display part 28, the driving circuitry 30 and the LC layer 32, separated by the dichroic mirror (DM) 34 and a semi-transparent conductive electrode (SCE) 36, advantageously based on grid structure. In its use as a three-way ITD device, this structure allows imaging to be formed on two opposite surfaces of the imager, front and back. In the case of a two-way ITD, either front side imaging (eye tracking) or back-side imaging (scene imaging) can be implemented. The back-side (bottom part of FIG. 2) imaging detects the scene information, whereas the front side (top part of FIG. 2) detects the eye image. The DM 34 and the SCE 36 allow the transmission of the NIR image carrying the eye image information to the imaging part. Due to the longer wavelength of the NIR radiation, the photo-carriers resulting from the absorption of the illuminated NIR beam are generated deep in the silicon substrate 38, much beyond the photodiode 26 line. In a uniform Si structure, most of these carriers would have been lost (recombined) before diffusing back to the photodiodes 26. Due to the particular use of the deep p-well structure (DPWS) 40, however, most of these photo-carriers will be quickly drifted by the potential difference generated by DPWS 40, back into the photodiodes 26, thereby generating the desired photo-current, with relatively low losses. The DM 34 acts to reflect the visible beam, which double-passes the LC layer 32, to pick up the spatially rotated polarization modulation image. This DM further allows the transmission of the NIR radiation into the substrate 38 and the photodiode 26.

In addition to the efficient channelling effect of the DPWS 40 on the NIR-photo-generated carriers, this structure also helps to substantially reduce the cross-talk between pixels, as well as the photo-activation of the electronic (LC driver and imager) circuits.

Turning back to FIG. 1, as seen, the beam is transmitted by the DM 36 of the ITD 8, which transmits NIR radiation beam but reflects visible radiation. It is also partially transmitted by the SCE 36. The transmitted NIR radiation beam is finally absorbed by the imaging part 24 of the ITD 8, thus delivering the retinal imagery for eye-tracking purposes.

Visible radiation from the visible LED 42 passes through a condenser lens 44, is S-polarized by the sheet polarizer 46, and reflected by the PBS 20 into the ITD 8. The reflected radiation reads out the image information (which is the processed input imagery) being reflected from the DM 34 of the ITD 8, thus double-passing the LC layer 32, which spatially modulates its polarization according to the signal pattern. The effect of the LC layer 32 modulation is to selectively rotate the polarization in each pixel of the image information signal pattern, in proportion to the input imagery at the display part of the ITD 8. Thus, the polarization-rotated imagery (now R-polarized) is transmitted by the PBS 20, and then reflected by the NBS 14 into the retina, which is the second focal plane of this beam (the first being the LC plane of the ITD 8).

The device thus accomplishes the four functions of see-through, scene-imaging, eye-imaging and processed imagery display by two separate devices: (a) the scene imager 6 and (b) the ITD 8. The feature to note here is the common optical path shared by the two functions of eye-imaging and display of the eye-tracking ITD. This requires that the retina will constitute the first focal plane for both beams (NIR 10, 10′ and VIS 42), while the second focal planes (the LC 32 for the VIS 42 and the imager of the ITD 8 for the NIR 10,10′) be within the depth of focus from each other. This is physically possible in this arrangement since the LC layer 32 is located very close, e.g., within 1-2 micrometers from the imaging plane of the ITD 8.

This arrangement is not only novel in its optical path sharing, but it also employs imaging of the retina, rather than imaging of the pupil, which is the common method of eye-tracking, in order to determine the eye-ball position. The path-sharing method is important for miniaturizing the goggle size.

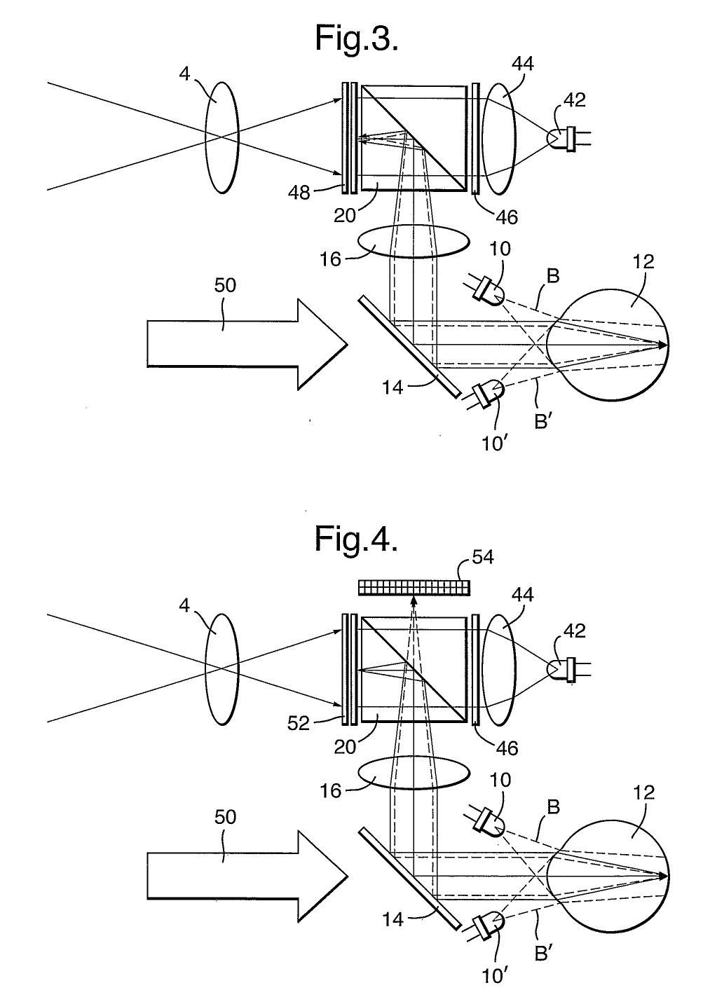

The embodiment of FIG. 3 illustrates a 3-way ITD 48. This arrangement allows the three functions of ambient scene and eye imaging, as well as the display to be performed using a single 3-Way ITD chip described above. This, in addition to the see-through capability via the see-through port 50, e.g., a spherical semi- transparent magnifier, which is provided in all HM devices such as goggles, is considered a preferred choice for the low vision applications, although non-see-through design options are also possible utilizing the ITD 8.

Referring also to FIG. 3, here the S-polarized NIR radiation beam again illuminates the retina, the reflected image of which is reflected again by the NBS 14 into the PBS 20, which, with its S-polarization reflection arrangement, reflects the NIR into the front side of the 3-way ITD 48. The NIR radiation beam is transmitted by the DM 34, as well as by the SCE 36 and is imaged by the imager part of the ITD. The R-polarized VIS beam is transmitted by the PBS 20, but is reflected by the ITD's DM 34. It double-passes the LC layer and picks up the displayed image in the form of spatial polarization modulation by the LC. The rotated, S-polarized VIS beam, carrying the display information, is reflected by the (S-reflecting) PBS 20 into the NBS 14, which causes it to be reflected to the retina as the second focal plane, where it is imaged, thus accomplishing the display function. Finally, the ambient scene is imaged into the backside part of the 3-way ITD 48 and into the imager part (photo-diode array), where it is detected. Since imaging of the eye requires substantially lower resolution (in the order of 100×100 pixels), as compared to the scene imaging requirement (which requires well over 1000×1000 pixels), the 3-way ITD allows the complex 2-sided imaging to be performed, by compromising the imaging resolution quality in one of the channels (eye-tracking), while maintaining high resolution in the other (scene imaging). In order to avoid the simultaneous imaging of both eye and scene imagery, the NIR beams can be pulsed in synchronization with the scene imager such that only a single type of imaging is performed at any particular time.

A modification of the embodiments of FIG. 1 and FIG. 3 is illustrated in FIG. 4, showing a separate eye tracker utilizing two separate elements for performing the three functions of scene, eye imaging and display (as well as the fourth function of see-through). Here, however, the ITD 52 is used for the input scene imaging and for the display of the processed image, whereas the second imager 54 is used solely for the eye tracking (imaging) purpose.

In this embodiment the R-polarized NIR Beam path is identical to the one described with reference to FIG. 1. The visible beam is initially R-polarized (as opposed to being initially S-polarized in FIG. 1). It is transmitted by the PBS 20 into the ITD 8, reflected by the DM 34 of the ITD, and double-passes the LC layer 32, thus carrying the spatially-rotated polarization modulation (display) information of the image. The rotated, S-polarization imagery is reflected by the PBS 20 into the shared optical path, where again it is reflected by the second beam splitter into the retina, as the second focal plane, thereby being imaged by the eye. The advantage of this arrangement relative to the embodiment of FIG. 1 is that the ITD can be fully exploited in terms of its resolution, for scene imagery which requires significantly high resolution, relative to the use as an eye imager which requires a relatively low resolution.

The method of implementing the required gaze-controlled retinal mapping for a specific low vision patient (LVP), is as follows:

The LVP is first clinically evaluated and the patient's retinal functionality is thoroughly mapped to determine the most effective region of a preferred retinal locus (PRL). This information is then stored in the low-vision goggle (LVG) processor, to determine the direction relative to the patient's direction of gaze (DoG) to which the acquired image should be displayed. The DoG is determined by the instantaneous output of the eye-tracker, which is part of the LVG system. The LVG processor then computes the retinal co-ordinates based on the PRL information and the DoG output. The processor next determines the appropriate image offset on the display part of the ITD, such that the required image will be displayed to the appropriate retinal location, corresponding to the patient's PRL.

It will be evident to those skilled in the art that the invention is not limited to the details of the foregoing illustrated embodiments and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A low-vision aid system, comprising:
a scene imager;
means for illuminating a retina of an eye with near IR (NIR) radiation to be reflected from the retina of the eye to an eye tracking, imager;
an image transceiver device (ITD) having means capable of providing eye tracking imaging and enhanced scene display,
said display having an array of pixels for driving a liquid crystal layer while selectively rotating the polarization of individual pixels of said array of pixels, to allow the transference of selected portions of said display to reach the retina of the eye.

2. The system as claimed in claim 13 wherein said enhanced scene display imager and the eye tracking imager are a single ITD unit.

3. The system as claimed in claim 1, wherein said ITD includes a deep P-well structure (DPWS) for enhancing the collection of photo-carriers generated in a silicon substrate, displaced from photo-diodes.

4. The system as claimed in claim 1, wherein said ITD includes a dichoric mirror allowing said NIR radiation to be transmitted while reflecting visible radiation impinging thereon.

5. The system as claimed in claim 1, wherein said ITD includes a semi-transparent conductive electrode (SCE) allowing NIR radiation to be transmitted therethrough.

6. The system as claimed in claim 1, wherein NIR radiation reflected from the retina is further reflected by a beam splitter disposed in front of said eye, to a polarizing cube beam splitter for transmitting polarized light to said ITD.

7. The system as claimed in claim 6, further comprising a source of visible radiation transmitting said radiation through a polarizer to the polarizing cube beam splitter to impinge on said ITD.

8. The system as claimed in claim 6, wherein said ITD is a 3-way ITD allowing simultaneous scene imaging, eye imaging and enhanced scene display, said ITD being disposed between the polarizing cube beam splitter and the scene to be imaged.

9. The system as claimed in claim 6, wherein said ITD, operating as a combined back illuminated scene imager and enhanced scene display, is disposed between the polarizing cube beam splitter and the scene to be imaged and further comprising an eye tracking imager axially disposed with respect to the NIR radiation reflected by said beam splitter adjacent to, and beyond, said polarized cube beam splitter.

10. The system as claimed in claim 1, wherein said ITD includes a LC layer and an array of photodiodes forming two adjacently disposed image and object planes for displaying an enhanced scene image to the eye and for performing eye imaging, using a shared optical path.

11. The system as claimed in claim 1, further comprising a see-through port.

12. The system as claimed in claim 6, further comprising a polarizer for polarizing a visible light beam allowing its transmission by said polarizing cube beam splitter to the display of the system.
